(19) United States
(12) Patent Application Publication
(10) Pub. No.: US 2026/0159467 A1
(43) Pub. Date: Jun. 11, 2026

(54) PROCESS FOR CONDENSING OXYGENATE MIXTURES

(71) Applicant: Topsoe A/S, Kgs. Lyngby (DK)

(72) Inventors: Matthias Josef BEIER, Birkerød (DK); Max THORHAUGE, Herlev (DK); Morten Boberg LARSEN, Smørum (DK); Christian Mårup OSMUNDSEN, Gentofte (DK)

(73) Assignee: Topsoe A/S, Kgs. Lyngby (DK)

(21) Appl. No.: 18/878,117

(22) PCT Filed: Jul. 7, 2023
(86) PCT No.: PCT/EP2023/068853

§ 371 Date: Dec. 23, 2024

(30) Foreign Application Priority Data
Jul. 8, 2022 (EP) 22183766.9
Jul. 8, 2022 (EP) 22183770.1

(51) Int. Cl. C07C 45/45; B01D 19/04; C07C 45/81

(52) U.S. Cl. CPC C07C 45/45; B01D 19/0409; C07C 45/81

(57) ABSTRACT

There is provided a process for the at least partial condensation of an oxygenate mixture the process comprising the steps of: (a) providing a vapour phase oxygenate mixture obtained from fragmentation of an aqueous solution of carbohydrates; (b) performing on the vapour phase oxygenate mixture an at least partial condensation to provide a condensate; and (c) combining an antifoaming agent and the condensate.

### FIELD OF THE INVENTION

The present invention relates to a process and a system for the at least partial condensation of an oxygenate mixture to provide a condensate.

### BACKGROUND OF THE INVENTION

Biomass is of particular interest as a raw material due to its potential for supplementing and possibly replacing petroleum as a feedstock for the preparation of commercial chemicals. In recent years, various technologies for exploiting biomass have been investigated.

Carbohydrates represent a large fraction of biomass, and various strategies for their efficient use as a feedstock for the preparation of commercial chemicals are being established. These strategies include various fermentation-based processes, catalyst-based processes, pyrolysis, thermolytic fragmentation, and other processes, such as hydrogenolysis, hydroformylation or acid catalyzed dehydration.

The conversion of biomass by pyrolysis processes is desirable due to the high volumetric production rates which can be achieved, and due to the ability of these types of processes to convert a wide range of substrates to a small range of products. However, current pyrolysis processes typically have challenges when implementing them into industrial settings, where e.g. high efficiency and long-term stability of the processes are desired for industrial applicability.

One pyrolysis method for converting carbohydrates, and in particular sugars, into commercially interesting chemicals is “thermolytic fragmentation”. It may be followed by further process steps. It may also be referred to as “hydrous thermolysis” or “carbohydrate cracking”.

Examples of chemicals produced from biomass include: substitute natural gas, biofuels, such as ethanol and bio-diesel, food browning materials, and commercial chemicals, such as diols (ethylene glycol, and propylene glycol), acids (lactic acid, acrylic acid, and levulinic acid) and a wide range of other important chemical intermediates (epichlorohydrin, isoprene, furfural, and synthesis gas).

Accordingly, new uses of C1-C3 oxygenate products are being developed and an increasing demand for those products is expected. Such oxygenate products may e.g. be used for producing ethylene glycol and propylene glycol by subjecting the oxygenate product to hydrogenation (see e.g. WO 2016/001169) or for scavenging hydrogen sulphide (see e.g. WO 2017/064267). However, many other uses may be envisaged.

Upon fragmentation of carbohydrates, compositions consisting primarily of C1-C3 oxygenates are formed. The primary C1 oxygenate is formaldehyde, which is undesirable in many products because it is highly toxic/carcinogenic, and has been shown to act as a catalyst poison (see US 2016/002137). The primary C2 oxygenate is glycolaldehyde, which is a desired product as it may be converted to useful chemicals, such as ethylene glycol, glycolic acid and methyl vinylglycolate (methyl 2-hydroxy-3-butenoate). Oxygenate mixtures produced from the fragmentation of carbohydrates are useful in a number of different applications, where the toxicity of formaldehyde may be a problem. Preparation of a formaldehyde free, or depleted, composition is therefore highly desirable. US 2016/002137 discusses a method for removing formaldehyde by reactive distillation; however, this method adds additional process steps.

WO 02/40436 discloses a method for the production of glycolaldehyde by hydrous thermolysis. The method comprises: preparing an aqueous sugar solution; atomizing the aqueous sugar solution; injecting the atomised aqueous sugar solution into a reactor heated between 500 and 600° C., creating a vaporous pyrolysis product; cooling the vaporous pyrolysis product in a condenser, obtaining a liquid condensate; collecting the liquid condensate into a holding tank to yield a glycolaldehyde-rich liquid; and filtering the glycolaldehyde-rich liquid.

It would be desirable to provide an alternative or improved process and/or system for production of an oxygenate mixture obtained from fragmentation of an aqueous solution of carbohydrates. It also would be desirable for said production to be suitable for industrial scale.

### SUMMARY OF THE INVENTION

In one aspect there is provided a process for the at least partial condensation of an oxygenate mixture, the process comprising the steps of:
(a) providing a vapour phase oxygenate mixture obtained from fragmentation of an aqueous solution of carbohydrates;
(b) performing on the vapour phase oxygenate mixture an at least partial condensation to provide a condensate; and
(c) combining an antifoaming agent and the condensate.

In one aspect there is provided a composition prepared by a process comprising the steps of:
(a) providing a vapour phase oxygenate mixture obtained from fragmentation of an aqueous solution of carbohydrates;
(b) performing on the vapour phase oxygenate mixture an at least partial condensation to provide a condensate; and
(c) combining an antifoaming agent and the condensate.

In one aspect there is provided a condensate prepared by a process comprising the steps of:
(a) providing a vapour phase oxygenate mixture obtained from fragmentation of an aqueous solution of carbohydrates;
(b) performing on the vapour phase oxygenate mixture an at least partial condensation to provide a condensate; and
(c) combining an antifoaming agent and the condensate.

In one aspect there is provided a system configured to at least partially condense an oxygenate mixture, the system comprising:
(a) a fragmentation reactor configured to fragment an aqueous solution of carbohydrates to provide a vapour phase oxygenate mixture;
(b) a condenser configured to at least partially condense the vapour phase oxygen-ate mixture to provide a condensate; and
(c) a unit configured to combine an antifoaming agent and the condensate.

### BRIEF DESCRIPTION OF THE DRAWINGS

Embodiments of the present invention are explained by way of examples and with reference to the accompanying drawings. The appended drawings illustrate only examples of embodiments of the present invention, and they are therefore not to be considered limiting of its scope, as the invention may admit to other alternative embodiments.

FIG. 1 shows foam height data from example 1; and

FIG. 2 is a schematic drawing of the condensation process of example 2.

### DETAILED DESCRIPTION OF THE INVENTION

### Process

As discussed herein, in one aspect there is provided a process for the at least partial condensation of an oxygenate mixture, the process comprising the steps of:
(a) providing a vapour phase oxygenate mixture obtained from fragmentation of an aqueous solution of carbohydrates;
(b) performing on the vapour phase oxygenate mixture an at least partial condensation to provide a condensate; and
(c) combining an antifoaming agent and the condensate.

We have found that performing on the vapour phase oxygenate mixture an at least partial condensation to provide a condensate creates a significant amount of foam along with the condensate. In an industrial-scale process, the foam is problematic for the condenser and/or units downstream of the condenser that typically are not configured to handle foam.

We also have found that by combining an antifoaming agent and the condensate to reduce or suppress foam, one may provide a process for producing an oxygenate mixture obtained from fragmentation of an aqueous solution of carbohydrates which is suitable for industrial-scale production.

In one aspect, the process is a continuous process, which is normally preferred in industrial scale processes.

### Step (a)—Providing a Vapour Phase Oxygenate Mixture Obtained from Fragmentation of an Aqueous Solution of Carbohydrates

As discussed herein, the present process requires step (a) providing a vapour phase oxygenate mixture obtained from fragmentation of an aqueous solution of carbohydrates.

The fragmentation of the aqueous solution of carbohydrates may be achieved by any suitable means. In one aspect, the fragmentation of the aqueous solution of carbohydrates is thermolytic fragmentation. In one aspect, the fragmentation of the aqueous solution of carbohydrates is pyrolysis. The fragmentation of the aqueous solution of carbohydrates may be performed by any suitable process. In one aspect the fragmentation of the aqueous solution of carbohydrates is performed as described in WO2020/016209 or as in WO2017/216311.

Thermolytic fragmentation is in the present context meant to refer to a selective decomposition of carbohydrates into an oxygenate mixture brought about by heating the sugar to intermediate temperatures (400-600 degrees C) under inert conditions and with very short residence time. The employed heating rate is very high (>1000 degrees C/s) and the residence time low (<1 s) to minimize the selectivity to polymerization products or permanent gases. An important chemical compound formed from thermolytic fragmentation of sugars is glycolaldehyde (hydroxyacetaldehyde). Glycolaldehyde is the smallest compound known which contain both a hydroxy and a carbonyl group, and it may be referred to as a sugar compound. It is highly reactive and is a useful platform chemical for making other chemicals such as ethylene glycol and glycolic acid. It is known to be an unstable molecule at elevated temperatures. See e.g. EP 0158517 B1, which recommends low temperature vacuum distillation for purifying glycolaldehyde.

Upon thermolytic fragmentation of the carbohydrates, a composition (i.e. a vapour phase oxygenate mixture) consisting primarily of C1-C3 oxygenates is formed. Besides the main product, namely the C2 oxygenate glycolaldehyde, the product obtained by fragmentation (e.g. thermolysis) of carbohydrates also contains varying amounts of C1 to C3 oxygenates, such as formaldehyde, glyoxal, pyruvaldehyde and acetol as well as minor amounts of larger molecules. The presence of the C1 oxygenate, formaldehyde, is often undesired and for some applications formaldehyde removal is necessary. The presence of the larger (and heavier) molecules in the condensate of the pyrolysis product is also undesired. Typically, significant resources may be spent on fractionation by distillation of a condensed oxygenate mixture, such as a glucose based pyrolysis product. This separation may produce an oxygenate syrup rich in glycolaldehyde and free of high-boiling byproducts.

In one aspect, the thermolytic fragmentation step is incorporated in the present process. In one aspect, the present invention comprises the step of thermolytic fragmentation of an aqueous solution of carbohydrates to provide the vapour phase oxygenate mixture of step (a). In one aspect, the step (b) is performed on a vapour phase oxygenate mixture obtained directly from thermolytic fragmentation of an aqueous solution of carbohydrates.

In one aspect, the thermolytic fragmentation of the aqueous solution of carbohydrates to provide the vapour phase oxygenate mixture comprises adding the aqueous solution of carbohydrates to a thermolytic fragmentation reactor.

In one aspect, the aqueous solution of carbohydrates is added to the thermolytic fragmentation reactor at a rate of at least 0.001 kg/hour, or at least 0.01 kg/hour, or at least 0.1 kg/hour, or at least 1 kg/hour, or at least 2 kg/hour, or at least 5 kg/hour, or at least 8 kg/hour, or at least 10 kg/hour, or at least 12 kg/hour, or at least 15 kg/hour.

In one aspect, the aqueous solution of carbohydrates is added to the thermolytic fragmentation reactor at a rate of no greater than 150 kg/hour, or no greater than 140 kg/hour, or no greater than 130 kg/hour, or no greater than 120 kg/hour, or no greater than 110 kg/hour, or no greater than 100 kg/hour, or no greater than 90 kg/hour, or no greater than 80 kg/hour, or no greater than 70 kg/hour, or no greater than 60 kg/hour.

In one aspect, the aqueous solution of carbohydrates is added to the thermolytic fragmentation reactor at a rate of from 0.001 kg/hour to 150 kg/hour, or 0.01 kg/hour to 140 kg/hour, or 0.1 kg/hour to 130 kg/hour, or 1 kg/hour to 120 kg/hour, or 2 kg/hour to 110 kg/hour, or 5 kg/hour to 100 kg/hour, or 8 kg/hour to 90 kg/hour, or 10 kg/hour to 80 kg/hour, or 12 kg/hour to 70 kg/hour, or 15 kg/hour to 60 kg/hour.

In one aspect, the thermolytic fragmentation of the aqueous solution of carbohydrates to provide the vapour phase oxygenate mixture comprises adding a flushing gas to the thermolytic fragmentation reactor. The flushing gas may comprise or consist of a fluidisation gas. Those skilled in the art will be familiar with the function of a fluidization gas, and understand that the fluidisation gas can be used to fluidise heat carrying particles (e.g. sand) in the fragmentation reactor.

In one aspect, the flushing gas is inert. In one aspect, the flushing gas may comprise air or nitrogen or steam. Suitable flushing gases are known to those skilled in the art.

The carbohydrates of the aqueous solution of carbohydrates may be any suitable carbohydrates. In one aspect, the carbohydrates of the aqueous solution of carbohydrates are selected from monosaccharides, disaccharides and mixtures thereof. In one aspect, the carbohydrates of the aqueous solution of carbohydrates are at least monosaccharides. In one aspect, the carbohydrates of the aqueous solution of carbohydrates are selected from the group consisting of sucrose, xylose, arabinose, mannose, tagatose, galactose, glucose, fructose, inulin, amylopectin (starch). In one aspect the carbohydrates of the aqueous solution of carbohydrates are at least glucose. In one aspect, the aqueous solution of carbohydrates is a sugar syrup.

In one aspect, the carbohydrates of the aqueous solution of carbohydrates comprise at least 20 wt. % monosaccharides based on the total amount of carbohydrates. In one aspect, the carbohydrates of the aqueous solution of carbohydrates comprise at least 30 wt. % monosaccharides based on the total amount of carbohydrates. In one aspect, the carbohydrates of the aqueous solution of carbohydrates comprise at least 40 wt. % monosaccharides based on the total amount of carbohydrates. In one aspect, the carbohydrates of the aqueous solution of carbohydrates comprise at least 50 wt. % monosaccharides based on the total amount of carbohydrates. In one aspect, the carbohydrates of the aqueous solution of carbohydrates comprise at least 60 wt. % monosaccharides based on the total amount of carbohydrates. In one aspect, the carbohydrates of the aqueous solution of carbohydrates comprise at least 70 wt. % monosaccharides based on the total amount of carbohydrates. In one aspect, the carbohydrates of the aqueous solution of carbohydrates comprise at least 80 wt. % monosaccharides based on the total amount of carbohydrates. In one aspect, the carbohydrates of the aqueous solution of carbohydrates comprise at least 90 wt. % monosaccharides based on the total amount of carbohydrates. In one aspect, the carbohydrates of the aqueous solution of carbohydrates comprise at least 95 wt. % monosaccharides based on the total amount of carbohydrates.

In one aspect, the carbohydrates of the aqueous solution of carbohydrates comprise at least 20 wt. % glucose based on the total amount of carbohydrates. In one aspect, the carbohydrates of the aqueous solution of carbohydrates comprise at least 30 wt. % glucose based on the total amount of carbohydrates. In one aspect, the carbohydrates of the aqueous solution of carbohydrates comprise at least 40 wt. % glucose based on the total amount of carbohydrates. In one aspect, the carbohydrates of the aqueous solution of carbohydrates comprise at least 50 wt. % glucose based on the total amount of carbohydrates. In one aspect, the carbohydrates of the aqueous solution of carbohydrates comprise at least 60 wt. % glucose based on the total amount of carbohydrates. In one aspect, the carbohydrates of the aqueous solution of carbohydrates comprise at least 70 wt. % glucose based on the total amount of carbohydrates. In one aspect, the carbohydrates of the aqueous solution of carbohydrates comprise at least 80 wt. % glucose based on the total amount of carbohydrates. In one aspect, the carbohydrates of the aqueous solution of carbohydrates comprise at least 90 wt. % glucose based on the total amount of carbohydrates. In one aspect, the carbohydrates of the aqueous solution of carbohydrates comprise at least 95 wt. % glucose based on the total amount of carbohydrates.

The aqueous solution of carbohydrates may comprise carbohydrates in any suitable amount. In one aspect, the aqueous solution of carbohydrates comprises carbohydrates in an amount of at least 10 wt. % based on the aqueous solution. In one aspect, the aqueous solution of carbohydrates comprises carbohydrates in an amount of at least 20 wt. % based on the aqueous solution. In one aspect, the aqueous solution of carbohydrates comprises carbohydrates in an amount of at least 30 wt. % based on the aqueous solution. In one aspect, the aqueous solution of carbohydrates comprises carbohydrates in an amount of at least 40 wt. % based on the aqueous solution. In one aspect, the aqueous solution of carbohydrates comprises carbohydrates in an amount of at least 50 wt. % based on the aqueous solution. In one aspect, the aqueous solution of carbohydrates comprises carbohydrates in an amount of at least 60 wt. % based on the aqueous solution. In one aspect, the aqueous solution of carbohydrates comprise glucose in an amount of at least 10 wt. % based on the aqueous solution. In one aspect, the aqueous solution of carbohydrates comprise glucose in an amount of at least 20 wt. % based on the aqueous solution. In one aspect, the aqueous solution of carbohydrates comprise glucose in an amount of at least 30 wt. % based on the aqueous solution. In one aspect, the aqueous solution of carbohydrates comprise glucose in an amount of at least 40 wt. % based on the aqueous solution. In one aspect, the aqueous solution of carbohydrates comprise glucose in an amount of at least 50 wt. % based on the aqueous solution. In one aspect, the aqueous solution of carbohydrates comprise glucose in an amount of at least 60 wt. % based on the aqueous solution.

For the aqueous solutions of carbohydrates the upper limits of carbohydrates are defined by the solubility of each carbohydrate. Both for carbohydrates, disaccharides, and monosaccharides, as well as any of the sugars (in particular glucose) an upper limit of 95 wt. %, such as 96, 97, 98, 99 wt. % of carbohydrate, based on the aqueous solution may be envisaged.

We have found that the at least partial condensation of vapour phase oxygenate mixtures derived from biomass is particularly prone to creating a significant amount of foam. In one aspect, the vapour phase oxygenate mixture is derived from biomass. In one aspect, the aqueous solution of carbohydrates is derived from biomass. In one aspect, the carbohydrates of the aqueous solution of carbohydrates are derived from biomass. In one aspect, the biomass is selected from plant biomass, animal biomass, or a combination thereof. Non-limiting examples of biomass include wood, wood residues, forestry residues, agricultural residues (e.g. straw, stover, corn, wheat, cane trash, and green agricultural wastes), agro-industrial waste (e.g. sugarcane bagasse, sugar beets and rice husk), animal waste (e.g. cow manure, and poultry litter), industrial waste (e.g. black liquor from paper manufacturing), sewage, municipal solid waste, and food processing waste.

As will be appreciated by a person skilled in the art, the vapour phase oxygenate mixture will have a mass ratio of components (e.g. glycolaldehyde to formaldehyde) which may vary depending on the nature of the aqueous solution of carbohydrates (the carbohydrate feed) and the conditions of the fragmentation.

In one aspect, the vapour phase oxygenate mixture comprises C1-C3 oxygenates. In one aspect, the vapour phase oxygenate mixture comprises at least 40 wt. % of C 1-C3 oxygenates based on the total weight of the vapour phase oxygenate mixture. In one aspect, the vapour phase oxygenate mixture comprises at least 50 wt. % of C 1-C3 oxygenates based on the total weight of the vapour phase oxygenate mixture. In one aspect, the vapour phase oxygenate mixture comprises at least 60 wt. % of C 1-C3 oxygenates based on the total weight of the vapour phase oxygenate mixture. In one aspect, the vapour phase oxygenate mixture comprises at least 70 wt. % of C 1-C3 oxygenates based on the total weight of the vapour phase oxygenate mixture.

There is no upper limit for which the invention will not work. However an upper limit of 90 wt. % of C1-C3 oxygenates based on the total weight of the vapour phase oxygenate mixture may be envisaged for any of the lower values given above.

In one aspect, the vapour phase oxygenate mixture consists essentially of C1-C3 oxygenates. In one aspect, the vapour phase oxygenate mixture substantially consists of C1-C3 oxygenates.

### Step (b)—Performing on the Vapour Phase Oxygenate Mixture an at Least Partial Condensation to Provide a Condensate

As discussed herein, the present process requires the step (b) of performing on the vapour phase oxygenate mixture an at least partial condensation to provide a condensate.

The present process covers partial condensation of the vapour phase oxygenate mixture. By “partial condensation”, we mean that a part of the vapour phase oxygenate mixture is condensed in the condensation step to provide the condensate. Where the condensation is a partial condensation, the condensate therefrom may be referred to as a “partial condensation condensate”. The present process covers total condensation of the vapour phase oxygenate mixture to provide the condensate. By “total condensation”, we mean that substantially all of the vapour phase oxygenate mixture is condensed in the condensation step to provide the condensate. Where the condensation is a total condensation, the condensate therefrom may be referred to as a “total condensation condensate”. It will be appreciated by a person skilled in the art that partial condensation and total condensation may result in condensates having different compositions.

In one aspect, there is provided a process for the total condensation of an oxygenate mixture, the process comprising the steps of:
(a) providing a vapour phase oxygenate mixture obtained from fragmentation of an aqueous solution of carbohydrates;
(b) performing on the vapour phase oxygenate mixture a total condensation to provide a total condensate; and
(c) combining an antifoaming agent and the condensate.

As will be appreciated by a person skilled in the art, the vapour phase oxygenate mixture may have a ratio of glycolaldehyde to formaldehyde which may vary depending on the nature of the aqueous solution of carbohydrates (the carbohydrate feed) and the conditions of the fragmentation.

In one aspect, the vapour phase oxygenate mixture has a mass ratio of glycolaldehyde to formaldehyde of 2:1 to 18:1. In one aspect, the vapour phase oxygenate mixture has a mass ratio of glycolaldehyde to formaldehyde of 3:1 to 18:1. In one aspect, the vapour phase oxygenate mixture has a mass ratio of glycolaldehyde to formaldehyde of 4:1 to 18:1. In one aspect, the vapour phase oxygenate mixture has a mass ratio of glycolaldehyde to formaldehyde of 5:1 to 18:1. In one aspect, the vapour phase oxygenate mixture has a mass ratio of glycolaldehyde to formaldehyde of 2:1 to 15:1. In one aspect, the vapour phase oxygenate mixture has a mass ratio of glycolaldehyde to formaldehyde of 3:1 to 15:1. In one aspect, the vapour phase oxygenate mixture has a mass ratio of glycolaldehyde to formaldehyde of 4:1 to 15:1. In one aspect, the vapour phase oxygenate mixture has a mass ratio of glycolaldehyde to formaldehyde of 5:1 to 15:1. In one aspect, the vapour phase oxygenate mixture has a mass ratio of glycolaldehyde to formaldehyde of 2:1 to 10:1. In one aspect, the vapour phase oxygenate mixture has a mass ratio of glycolaldehyde to formaldehyde of 3:1 to 10:1. In one aspect, the vapour phase oxygenate mixture has a mass ratio of glycolaldehyde to formaldehyde of 4:1 to 10:1. In one aspect, the vapour phase oxygenate mixture has a mass ratio of glycolaldehyde to formaldehyde of 5:1 to 10:1.

As will be appreciated by a person skilled in the art, the composition of the condensate may vary depending on whether a partial or total condensation is performed or a total condensation is performed. For example, if in step (b) a partial condensation is performed, the mass ratio of the components (e.g. glycolaldehyde to formaldehyde) of the vapour phase oxygenate mixture may be different from the mass ratio of the components of the condensate. By contrast, if in step (b) a total condensation is performed, mass ratio of the components (e.g. glycolaldehyde to formaldehyde) of the vapour phase oxygenate mixture may be substantially the same as the mass ratio of the components of the condensate.

In one aspect, the condensate has a mass ratio of glycolaldehyde to formaldehyde of at least 5:1. In one aspect, the condensate has a mass ratio of glycolaldehyde to formaldehyde of at least 10:1. In one aspect, the condensate has a mass ratio of glycolaldehyde to formaldehyde of at least 15:1. In one aspect, the condensate has a mass ratio of glycolaldehyde to formaldehyde of at least 20:1. In one aspect, the condensate has a mass ratio of glycolaldehyde to formaldehyde of at least 30:1. In one aspect, the condensate has a mass ratio of glycolaldehyde to formaldehyde of at least 40:1. In one aspect, the condensate has a mass ratio of glycolaldehyde to formaldehyde of at least 50:1. In one aspect, the condensate has a mass ratio of glycolaldehyde to formaldehyde of at least 60:1. In one aspect, the condensate has a mass ratio of glycolaldehyde to formaldehyde of at least 70:1. In one aspect, the condensate has a mass ratio of glycolaldehyde to formaldehyde of at least 80:1. In one aspect, the condensate has a mass ratio of glycolaldehyde to formaldehyde of at least 90:1. In one aspect, the condensate has a mass ratio of glycolaldehyde to formaldehyde of at least 100:1.

In one aspect, the condensate has a mass ratio of glycolaldehyde to formaldehyde of no greater than 500:1. In one aspect, the condensate has a mass ratio of glycolaldehyde to formaldehyde of no greater than 400:1. In one aspect, the condensate has a mass ratio of glycolaldehyde to formaldehyde of no greater than 300:1. In one aspect, the condensate has a mass ratio of glycolaldehyde to formaldehyde of no greater than 200:1. In one aspect, the condensate has a mass ratio of glycolaldehyde to formaldehyde of no greater than 180:1. In one aspect, the condensate has a mass ratio of glycolaldehyde to formaldehyde of no greater than 160:1. In one aspect, the condensate has a mass ratio of glycolaldehyde to formaldehyde of no greater than 150:1. In one aspect, the condensate has a mass ratio of glycolaldehyde to formaldehyde of no greater than 140:1. In one aspect, the condensate has a mass ratio of glycolaldehyde to formaldehyde of no greater than 130:1. In one aspect, the condensate has a mass ratio of glycolaldehyde to formaldehyde of no greater than 120:1. In one aspect, the condensate has a mass ratio of glycolaldehyde to formaldehyde of no greater than 110:1. In one aspect, the condensate has a mass ratio of glycolaldehyde to formaldehyde of no greater than 100:1.

In one aspect, the condensate has a mass ratio of glycolaldehyde to formaldehyde of from 10:1 to 200:1. In one aspect, the condensate has a mass ratio of glycolaldehyde to formaldehyde of from 10:1 to 180:1. In one aspect, the condensate has a mass ratio of glycolaldehyde to formaldehyde of from 10:1 to 160:1. In one aspect, the condensate has a mass ratio of glycolaldehyde to formaldehyde of from 10:1 to 150:1. In one aspect, the condensate has a mass ratio of glycolaldehyde to formaldehyde of from 10:1 to 140:1. In one aspect, the condensate has a mass ratio of glycolaldehyde to formaldehyde of from 10:1 to 130:1. In one aspect, the condensate has a mass ratio of glycolaldehyde to formaldehyde of from 10:1 to 120:1. In one aspect, the condensate has a mass ratio of glycolaldehyde to formaldehyde of from 10:1 to 110:1. In one aspect, the condensate has a mass ratio of glycolaldehyde to formaldehyde of from 10:1 to 100:1.

In one aspect, the condensate has a mass ratio of glycolaldehyde to formaldehyde of from 30:1 to 200:1. In one aspect, the condensate has a mass ratio of glycolaldehyde to formaldehyde of from 30:1 to 180:1. In one aspect, the condensate has a mass ratio of glycolaldehyde to formaldehyde of from 30:1 to 160:1. In one aspect, the condensate has a mass ratio of glycolaldehyde to formaldehyde of from 30:1 to 150:1. In one aspect, the condensate has a mass ratio of glycolaldehyde to formaldehyde of from 30:1 to 140:1. In one aspect, the condensate has a mass ratio of glycolaldehyde to formaldehyde of from 30:1 to 130:1. In one aspect, the condensate has a mass ratio of glycolaldehyde to formaldehyde of from 30:1 to 120:1. In one aspect, the condensate has a mass ratio of glycolaldehyde to formaldehyde of from 30:1 to 130:1. In one aspect, the condensate has a mass ratio of glycolaldehyde to formaldehyde of from 30:1 to 100:1.

The process parameters required to perform the at least partial condensation of the present invention can be readily determined by a person skilled in the art. Key parameters are the composition of the vapour phase oxygenate mixture, the at least partial condensation temperature, and the at least partial condensation pressure. In general, the composition of the vapour phase oxygenate mixture will be defined by the fragmentation process. Similarly, the pressure of the at least partial condensation typically may be defined by requirements of the upstream process. Thus, the temperature of the at least partial condensation is the parameter typically controlled to achieve the required separation. If the pressure is not defined by upstream process considerations, and can be freely controlled, in principle this may be used as the controlling parameter instead (at a fixed temperature). However for most practical applications, controlling the temperature of the at least partial condensation will be more appropriate. In any case, the considerations for using the pressure as controlling parameter will be identical to those outlined below for temperature.

In one aspect, the condensate is condensed at a temperature of from 0 to 150° C. In one aspect, the condensate is condensed at a temperature of from 10 to 150° C. In one aspect, the condensate is condensed at a temperature of from 20 to 150° C. In one aspect, the condensate is condensed at a temperature of from 30 to 150° C. In one aspect, the condensate is condensed at a temperature of from 30 to 130° C. In one aspect, the condensate is condensed at a temperature of from 0 to 90° C. In one aspect, the condensate is condensed at a temperature of from 5 to 90° C. In one aspect, the condensate is condensed at a temperature of from 30 to 90° C. In one aspect, the condensate is condensed at a temperature of from 40 to 90° C. In one aspect, the condensate is condensed at a temperature of from 30 to 70° C. In one aspect, the condensate is condensed at a temperature of from 40 to 60° C.

In one aspect, there is provided a process for the at least partial condensation of an oxygenate mixture, the process comprising the steps of:
(a) providing a vapour phase oxygenate mixture obtained from fragmentation of an aqueous solution of carbohydrates; and
(b) performing on the vapour phase oxygenate mixture an at least partial condensation to provide a condensate, wherein the condensate is condensed at a temperature of from 0 to 150° C.; and
(c) combining an antifoaming agent and the condensate.

In one aspect, the condensate exiting the condenser has a temperature of from 0 to 150° C. In one aspect, the condensate exiting the condenser has a temperature of from 10 to 150° C. In one aspect, the condensate exiting the condenser has a temperature of from 20 to 150° C. In one aspect, the condensate exiting the condenser has a temperature of from 30 to 150° C. In one aspect, the condensate exiting the condenser has a temperature of from 0 to 90° C. In one aspect, the condensate exiting the condenser has a temperature of from 5 to 90° C. In one aspect, the condensate exiting the condenser has a temperature of from 40 to 90° C. In one aspect, the condensate exiting the condenser has a temperature of from 30 to 70° C. In one aspect, the condensate exiting the condenser has a temperature of from 40 to 60° C.

In one aspect, there is provided a process for the at least partial condensation of an oxygenate mixture, the process comprising the steps of:
(a) providing a vapour phase oxygenate mixture obtained from fragmentation of an aqueous solution of carbohydrates; and
(b) performing on the vapour phase oxygenate mixture an at least partial condensation to provide a condensate, wherein the condensate formed by the at least partial condensation has a temperature of from 0 to 150° C.; and
(c) combining an antifoaming agent and the condensate.

As discussed herein, prior to step (b) an intermediate step may be performed on the oxygenate mixture. For example, an initial partial condensation may be performed on the oxygenate mixture prior to the partial condensation step (b). In one aspect, step (b) is performed on an oxygenate mixture obtained directly from fragmentation of an aqueous solution of carbohydrates. This intermediate step may be selected, for example, depending on the composition of the oxygenate mixture obtained directly from fragmentation of an aqueous solution of carbohydrates. As will be understood by one skilled in the art, the composition of the oxygenate mixture obtained directly from fragmentation of an aqueous solution of carbohydrates will depend, among other things, on the composition of the aqueous solution of carbohydrates and the fragmentation process parameters.

In one aspect, the performing on the vapour phase oxygenate mixture an at least partial condensation comprises: feeding an input stream comprising the vapour phase oxygenate mixture through (or into) a condenser to provide the condensate and an output stream. Where a partial condensation is used, the output stream may comprise the partial condensation vapour phase. In one aspect, the input stream comprises the flushing gas. The condensate and the output stream exit the condenser, e.g. at or towards the top of the condenser.

Downstream of the condenser, one or more environmentally problematic components (e.g. from the output stream) may be removed from gas phase using a catalyst. We have found that foam can compromise or destroy the catalyst. Through use of the antifoaming agent, the catalyst can be protected from the foam so as to maintain its performance.

In one aspect, the output stream exiting the condenser has a temperature of from 0 to 50° C. In one aspect, the output stream exiting the condenser has a temperature of from 0 to 40° C. In one aspect, the output stream exiting the condenser has a temperature of from 0 to 30° C. In one aspect, the output stream exiting the condenser has a temperature of from 0 to 20° C. In one aspect, the output stream exiting the condenser has a temperature of from 10 to 20° C.

In one aspect, the performing on the vapour phase oxygenate mixture an at least partial condensation comprises: feeding the input stream comprising the vapour phase oxygenate mixture and the flushing gas into the condenser to provide the condensate and the output stream, the output stream comprising the flushing gas.

In one aspect, the performing on the vapour phase oxygenate mixture an at least partial condensation comprises: recycling at least part of the output stream comprising the flushing gas into the input stream. We have found that recycling the output stream in an industrial scale process can result in a significant amount of foam being formed. However, by virtue of the antifoaming agent, the present invention allows for such recycling in an industrial scale process with reduced levels of foam (e.g. acceptable or minimal amounts of foam) being created.

In one aspect, the performing on the vapour phase oxygenate mixture an at least partial condensation comprises: splitting the condensate into at least a condensate product stream and a condensate recycle stream.

In one aspect, the mass ratio of the condensate recycle stream to the condensate product stream is from 5:1 to 50:1. In one aspect, the mass ratio of the condensate recycle stream to the condensate product stream is from 10:1 to 30:1. In one aspect, the mass ratio of the condensate recycle stream to the condensate product stream is from 10:1 to 20:1.

In one aspect, the performing on the vapour phase oxygenate mixture an at least partial condensation comprises: adding the condensate recycle stream to the condenser.

In one aspect, the condensate is cooled. In one aspect, the condensate recycle stream is cooled before the condensate recycle stream is added to the condenser. We have found that the adding the condensate (e.g. the condensate recycle stream) to the condenser promotes condensation of the vapour phase oxygenate mixture.

In one aspect, the condensate recycle stream added to the condenser has a temperature of from 0 to 50° C. In one aspect, the condensate recycle stream added to the condenser has a temperature of from 0 to 40° C. In one aspect, the condensate recycle stream added to the condenser has a temperature of from 0 to 30° C. In one aspect, the condensate recycle stream added to the condenser has a temperature of from 0 to 20° C. In one aspect, the condensate recycle stream added to the condenser has a temperature of from 10 to 20° C. In this context, the temperature at which the condensate recycle stream is added to the condenser can mean the temperature of the condensate recycle stream entering the condenser.

In one aspect, condensate recycle stream is added to the condenser at or towards the top of the condenser. In this way, the condensate flows through the condenser under gravity. In one aspect, condensate recycle stream is added to the condenser at or towards the bottom of the condenser.

In one aspect, the condenser comprises a packing material. The packing material increases the contact area between the condensate to be recycled, the antifoaming agent (as applicable), and the vapour phase oxygenate mixture (e.g. the input stream) in the condenser. Those skilled in the art will be aware of suitable packing materials.

In one aspect, the vapour phase oxygenate mixture (or the input stream) is filtered before the performing on the vapour phase oxygenate mixture an at least partial condensation.

### Step (c)—Combining an Antifoaming Agent and the Condensate

As discussed herein, the present process requires the step (c) of combining an antifoaming agent and the condensate.

In the context of the present invention, it will be understood that the antifoaming agent may eliminate existing foam, or prevent the formation of foam, or eliminate existing foam and prevent the formation of foam. Thus, in the context of the present invention, it will be understood that the antifoaming agent is capable of one or both of eliminating existing foam and preventing the formation of foam.

In one aspect, the combining the antifoaming agent and the condensate comprises adding the antifoaming agent to the condensate. In one aspect, the combining the antifoaming agent and the condensate comprises continuously or intermittently adding the antifoaming agent to the condensate.

In one aspect, the combining the antifoaming agent and the condensate comprises adding the condensate to the antifoaming agent. For example, the antifoaming agent may be contained by a vessel to which the condensate is added.

In one aspect, the combining the antifoaming agent and the condensate comprises combining the antifoaming agent and the condensate in the condenser. In one aspect, the combining the antifoaming agent and the condensate comprises adding the antifoaming agent to the condenser.

In one aspect, the combining the antifoaming agent and the condensate comprises adding the condensate recycle stream to the condenser.

In one aspect, the combining the antifoaming agent and the condensate comprises adding the antifoaming agent to the condensate recycle stream to form a mixture and adding the mixture to the condenser.

In one aspect, the mixture is split into a plurality of streams. In one aspect, the each of the plurality of streams is added to a respective part of the condenser. In one aspect, at least part of the mixture (e.g. by virtue of a stream) is added to the condenser at or towards the top of the condenser. In one aspect, at least part of the mixture is added (e.g. by virtue of a stream) to the condenser above the packing material. In one aspect, at least part of the mixture is added (e.g. by virtue of a stream) to the condenser at or towards the bottom of the condenser. In one aspect, at least part of the mixture is added (e.g. by virtue of a stream) to the condenser below the packing material. The references to “top”, “bottom”, “above”, and “below” with respect to gravity.

In one aspect, the at least part of the mixture added to the condenser flows in countercurrent to the flow of the vapour phase oxygenate mixture in the condenser.

In one aspect, the mixture added to the condenser has a temperature of from 0 to 50° C. In one aspect, the mixture added to the condenser has a temperature of from 0 to 40° C. In one aspect, the mixture added to the condenser has a temperature of from 0 to 30° C. In one aspect, the mixture added to the condenser has a temperature of from 0 to 20° C. In one aspect, the mixture added to the condenser has a temperature of from 10 to 20° C. In this context, the temperature at which the mixture is added to the condenser can mean the temperature of the mixture entering the condenser.

### Antifoaming Agent

In one aspect, the antifoaming agent is added to the condensate at a rate of at least 0.0000001 mL/hour, or at least 0.000001 mL/hour, or at least 0.00001 mL/hour, or at least 0.0001 mL/hour, or at least 0.001 mL/hour, or at least 0.01 mL/hour, or at least 0.1 mL/hour, or at least 1 mL/hour, or at least 1.2 mL/hour, or at least 1.5 mL/hour.

In one aspect, the antifoaming agent is added to the condensate at a rate of no greater than 200 mL/hour, or no greater than 100 mL/hour, or no greater than 50 ml/hour, or no greater than 20 mL/hour, or no greater than 10 mL/hour, or no greater than 8 mL/hour, or no greater than 6 mL/hour, or no greater than 5 mL/hour, or no greater than 4 mL/hour, or no greater than 3.8 mL/hour.

In one aspect, the antifoaming agent is added to the condensate at a rate of from 0.0000001 mL/hour to 200 mL/hour, or 0.000001 mL/hour to 100 ml/hour, or 0.00001 mL/hour to 50 mL/hour, or 0.0001 mL/hour to 20 mL/hour, or 0.001 mL/hour to 10 mL/hour, or 0.01 mL/hour to 8 mL/hour, or 0.1 mL/hour to 6 mL/hour, or 1 mL/hour to 5 mL/hour, or 1.2 mL/hour to 4 mL/hour, or 1.5 mL/hour to 3.8 mL/hour.

In one aspect, the mass ratio of the antifoaming agent to the condensate in the combined antifoaming agent and condensate is at least 1:1000000, or at least 1:800000, or at least 1:500000, or at least 1:200000, or at least 1:100000, or at least 1:80000, or at least 1:50000, or at least 1:25000, or at least 1:20000, or at least 1:18000.

In one aspect, the mass ratio of the antifoaming agent to the condensate in the combined antifoaming agent and condensate is no greater than 1:1, or no greater than 1:2, or no greater than 1:4, or no greater than 1:6, or no greater than 1:8, or no greater than 1:10, or no greater than 1:12, or no greater than 1:14, or no greater than 1:16, or no greater than 1:18.

In one aspect, the mass ratio of the antifoaming agent to the condensate in the combined antifoaming agent and condensate is from 1:1000000 to 1:1, or 1:800000 to 1:2, or 1:500000 to 1:4, or 1:200000 to 1:6, or 1:100000 to 1:8, or 1:80000 to 1:10, or 1:50000 to 1:12, or 1:25000 to 1:14, or 1:20000 to 1:16, or 1:18000 to 1:18.

In one aspect, the antifoaming agent has a surface tension at 20° C. of no greater than 70 mN/m, or no greater than 60 mN/m, or no greater than 50 mN/m, or no greater than 40 mN/m, or no greater than 35 mN/m, or no greater than 30 mN/m, or no greater than 25 mN/m, or no greater than 20 mN/m, or no greater than 15 mN/M, or no greater than 10 mN/m, or no greater than 5 mN/m.

In one aspect, the antifoaming agent has a surface tension at 20° C. of between 20 and 30 mN/m.

The surface tension may be measured using any suitable method, such as using surface tensiometer. In one aspect, the surface tension is measured using a Wilhelmy plate tensiometer, a Du Nouy ring tensiometer, or a bubble pressure tensiometer.

In one aspect, the antifoaming agent comprises one or more active ingredients. As would be understood by the skilled person, the “active ingredient(s)” of an antifoaming agent is the part of the antifoaming agent responsible for its antifoaming effect.

In one aspect, the antifoaming agent substantially comprises, consists essentially of, or consists of, the one or more active ingredients.

In one aspect, the antifoaming agent has a total active ingredient content of at least 0.0000001 wt. %, or at least 0.000001 wt. %, or at least 0.00001 wt. %, or at least 0.0001 wt. %, or at least 0.001 wt. %, or at least 0.01 wt. %, or at least 0.1 wt. %, or at least 0.2 wt. %, or at least 0.3 wt. %, or at least 0.4 wt. %, based on the total weight of the antifoaming agent.

In one aspect, the antifoaming agent has a total active ingredient content of up to 100 wt. %. In one aspect, the antifoaming agent has a total active ingredient content of no greater than 95 wt. %, or no greater than 90 wt. %, or no greater than 80 wt. %, or no greater than 70 wt. %, or no greater than 60 wt. %, or no greater than 50 wt. %, or no greater than 40 wt. %, or no greater than 30 wt. %, or no greater than 25 wt. %, or no greater than 20 wt. %, based on the total weight of the antifoaming agent.

In one aspect, the antifoaming agent has a total active ingredient content of from 0.0000001 wt. % to 100 wt. %, or 0.0000001 wt. % to 95 wt. %, or 0.000001 wt. % to 90 wt. %, or 0.00001 wt. % to 80 wt. %, or 0.0001 wt. % to 70 wt. %, or 0.001 wt. % to 60 wt. %, or 0.01 wt. % to 50 wt. %, or 0.1 wt. % to 40 wt. %, or 0.2 wt. % to 30 wt. %, or 0.3 wt. % to 25 wt. %, or 0.4 wt. % to 20 wt. %, based on the total weight of the antifoaming agent.

In one aspect, each of the one or more active ingredients is independently selected from an alcohol, an ether, a carboxylic acid, an ester, a silicone (e.g. an organosilicon), a silica, an oil (e.g. vegetable oil or mineral oil), a wax, and an acrylate. In one aspect, each of the one or more active ingredients is independently selected from an alcohol and a silicone.

In one aspect, where each of the one or more active ingredients is selected from an alcohol, the antifoaming agent may substantially comprise, consist essentially of, or consist of, the one or more active ingredients.

In one aspect, where each of the one or more active ingredients is selected from a silicone, the antifoaming agent may have at least 0.0000001 wt. %, or at least 0.000001 wt. %, or at least 0.00001 wt. %, or at least 0.0001 wt. %, or at least 0.001 wt. %, or at least 0.01 wt. %, or at least 0.1 wt. %, or at least 1 wt. %, or at least 5 wt. % of the silicone(s), based on the total weight of the antifoaming agent.

In one aspect, where each of the one or more active ingredients is selected from a silicone, the antifoaming agent may have no greater than 100 wt. %, or no greater than 95 wt. %, or no greater than 90 wt. %, or no greater than 80 wt. %, or no greater than 70 wt. %, or no greater than 60 wt. %, or no greater than 50 wt. %, or no greater than 40 wt. %, or no greater than 30 wt. %, or no greater than 20 wt. % of the silicone(s), based on the total weight of the antifoaming agent.

In one aspect, where each of the one or more active ingredients is selected from a silicone, the antifoaming agent may have from 0.0000001 wt. % to 100 wt. %, or 0.0000001 wt. % to 95 wt. %, or 0.000001 wt. % to 90 wt. %, or 0.00001 wt. % to 80 wt. %, or 0.0001 wt. % to 70 wt. %, or 0.001 wt. % to 60 wt. %, or 0.01 wt. % to 50 wt. %, or 0.1 wt. % to 40 wt. %, or 1 wt. % to 30 wt. %, or 5 wt. % to 20 wt. % of the silicone(s), based on the total weight of the antifoaming agent.

In one aspect, the antifoaming agent comprises an aqueous solution. In one aspect, the antifoaming agent comprises an aqueous solution of the one or more active ingredients.

In one aspect, the antifoaming agent comprises an emulsion. In one aspect, the antifoaming agent comprises a silicone-containing emulsion.

In one aspect, the alcohol is selected from a primary alcohol. In one aspect, the alcohol is selected from a secondary alcohol. In one aspect, the alcohol is selected from tertiary alcohol.

In one aspect, the alcohol is selected from a C1-C12 alcohol. In one aspect, the alcohol is selected from a C1-C10 alcohol. In one aspect, the alcohol is selected from a C1-C8 alcohol.

In one aspect, the alcohol is selected from a C1-C6 alcohol. In one aspect, the alcohol is selected from a C1-C4 alcohol. In one aspect, the alcohol is selected from a propanol. In one aspect, the alcohol is selected from 1-propanol and 2-propanol. In one aspect, the alcohol is 2-propanol.

In one aspect, the alcohol is selected from a polyalkylene glycol. In one aspect, the alcohol is polyethylene glycol.

In one aspect, the silicone is selected from a polydimethylsiloxane.

### Further Process Steps

The process of the present invention may comprise one or more further steps. These one or more further steps may be before, after, or intermediate to the steps recited herein.

In one aspect, the process comprises the further step of recovering the condensate (e.g. at least a part thereof, e.g. the condensate product stream). In one aspect, the condensate is recovered by distillation. In one aspect, the condensate is recovered by solvent extraction.

### Composition

In one aspect there is provided a composition prepared by the process described herein. The composition includes the condensate and the antifoaming agent.

### Condensate

In one aspect there is provided a condensate prepared by the process described herein.

### System

As described herein, in one aspect there is provided a system configured to at least partially condense an oxygenate mixture, the system comprising:
(a) a fragmentation reactor configured to fragment an aqueous solution of carbohydrates to provide a vapour phase oxygenate mixture;
(b) a condenser configured to at least partially condense the vapour phase oxygen-ate mixture to provide a condensate; and
(c) a unit configured to combine an antifoaming agent and the condensate.

In one aspect, the system is configured to a least partially condense the oxygenate mixture by performing the process described herein.

Suitable types of fragmentation reactor will be known to a person skilled in the art. In one aspect, the fragmentation reactor is a thermolytic fragmentation reactor. In one aspect, the fragmentation reactor is a pyrolytic fragmentation reactor. In one aspect, the fragmentation reactor is a fluidised bed reactor. In one aspect, the fluidisation bed reactor is selected from a bubbling bed reactor, a turbulent bed reactor, and a riser-type reactor.

In one aspect, the fragmentation reactor comprises one or more of a feed inlet; and a product outlet; a riser; and a fluidisation gas inlet.

Suitable condensers will be known to a person skilled in the art. In one aspect, the condenser is selected from a water-cooled condenser, an air-cooled condenser, and an evaporative condenser. In one aspect, the condenser is a cooling tower.

In one aspect, the system comprises a separator configured to separate particulate matter from the vapour phase oxygenate mixture. The separator may be a filter. The particulate matter may be dust, e.g. from the fragmentation reactor. The particulate matter may include heat carrying particles or fragments thereof from the fragmentation reactor, in aspects where heat carrying particles are used.

As described herein, the present invention covers partial condensation of the vapour phase oxygenate mixture to provide the condensate and/or total condensation of the vapour phase oxygenate mixture to provide the condensate. In one aspect, the system may comprise a single condenser. Where the system comprises a single condenser, the condenser may be used for total condensation of the vapour phase oxygenate mixture in step (b). In one aspect, the system may comprise multiple condensers. Where the system comprises multiple condensers, the condensers may be used for multiple partial condensations of the vapour phase oxygenate mixture in step (b).

Any suitable unit configured to combine the antifoaming agent and the condensate may be used.

In one aspect, the unit comprises a dispenser configured to dispense the antifoaming agent. In one aspect, the dispenser is configured to continuously or intermittently dispense the antifoaming agent. In one aspect, the dispenser is configured to continuously or intermittently dispense the antifoaming agent at a predetermined rate. Suitable rates of dispensing the condensate can be readily determined by a person skilled in the art. In one aspect, the dispenser is provided on the condenser. In one aspect, the dispenser is provided on each condenser.

In one aspect, the unit comprises a vessel containing the antifoaming agent. In one aspect, the condenser and the vessel are arranged in fluid communication. In one aspect, each condenser and the vessel are arranged in fluid communication. In this way, the condensate from the condenser may flow into the vessel containing the antifoaming agent to be combined with the antifoaming agent.

The present invention will now be described with reference to the following non-limiting examples.

### EXAMPLES

### Example 1—Effect of Antifoaming Agents on Foaming in Thermolytic Glucose Fragmentation Condensate

250 ml of a condensate obtained from thermolytic glucose fragmentation was obtained. The condensate comprised 8.3 g/L glyoxal, 12.8 g/L pyruvaldehyde (methylglyoxal), 89.3 g/L glycolaldehyde, 15.2 g/L formaldehyde, and 3.8 g/L acetol (hydroxyacetone) in water.

250 ml of the condensate was mixed with 12.5 mL of 2-propanol. The resulting mixture comprised 5 vol. % 2-propanol based on the total volume of the mixture. 50.0 ml of this mixture was added to a 100 mL measuring cylinder with a stopper. The height of the liquid (i.e. before shaking, see below) was noted. The 50.0 mL mixture in the measuring cylinder was shaken 30 times for 10 seconds in total. The total height of the liquid and any foam was noted. The height of the foam at 5 minutes after shaking was calculated as the difference between the total height 5 minutes after shaking and the height of the liquid (before shaking).

The above experiment was repeated using 1 vol. % 2-propanol based on the total volume of the mixture (i.e. rather than 5 vol. % 2-propanol).

The above experiment was also repeated using 20 ppmv Silfoam SE 47 (i.e. 0.002 wt. % Silfoam SE 47) based on the total volume of the mixture (i.e. rather than 5 vol. % 2-propanol). Silfoam SE47 is an oil in water emulsion based on polydimethylsiloxane and auxiliaries, and had an active ingredient concentration of 17 wt. %.

The above experiment was also repeated in the absence of 2-propanol.

Each of the above experiments was repeated in triplicate and average heights of the foam were calculated.

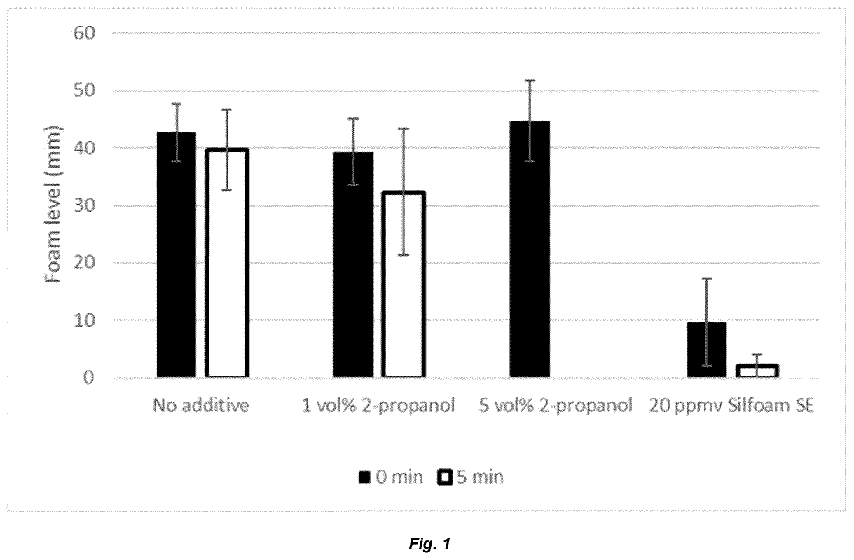

The results from the above experiments are illustrated in FIG. 1, in which the black bars represent the average difference between the height of the foam before shaking and immediately after shaking; and the white bars represent the average difference between the height of the foam before shaking and 5 minutes after shaking.

The results show that each of the antifoaming agents (2-propanol and Silfoam SE 47) had a positive effect on reducing and/or preventing the height of the foam.

### Example 2—Layout for a Continuous Condensation Process with Addition of Antifoaming Agent for Producing a Thermolytic Glucose Fragmentation Condensate

A glucose syrup feedstock solution (60 wt. % glucose and 40 wt. % water, based on the total weight of the feedstock solution) was subjected to thermolytic fragmentation using the system described in US10570078B2. This involved adding the glucose syrup feedstock solution into a fragmentation reactor which thermolytically fragmented the glucose syrup feedstock solution to form a product gas 101 (comprising a vapour phase oxygenate mixture) comprising C1-C3 oxygenates. The product gas 101 may also include flushing gas, which may comprise fluidisation gas from the fragmentation reactor.

The product gas 101 was directed through a first indirect cooling heat exchanger and then a filter to remove large particles, e.g. dust. In this particular example, the filter was a surface filtration device, although various other gas-solid filters (e.g. multicyclones) may be used. Other cooling means may also be used. In some examples, the first indirect heat exchanger and the filter may not be used.

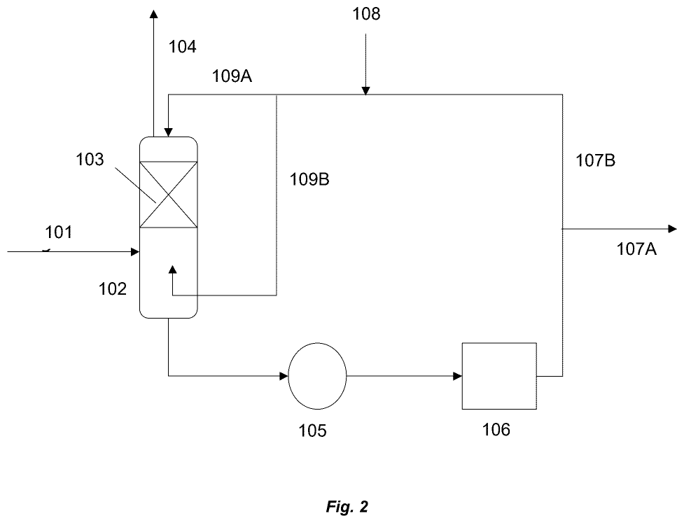

The product gas 101 exiting the filter was subjected to a condensation process, as illustrated in FIG. 2. This involved feeding the filtered product gas 101 into a condenser (in this example, a cooling tower) 102 comprising a packing material 103. A part of the product gas 101 flowing through the condenser 102 was condensed to form a condensate, which exited the bottom of the condenser 102 under gravity. The remaining part of the product gas 101, typically including CO and CO₂ (and possibly flushing gas), remained gaseous and exited the top of the condenser 102 through a gas outlet as a gas stream 104. The gas stream 104 exiting the gas outlet had a temperature of from 10 to 20° C., and the condensate exiting the condenser 102 had a temperature of from 40 to 60° C.

The condensate exiting the condenser 102 was directed to a pump 105 and then through an indirect plate heat exchanger 106, which cooled the condensate to a temperature of from 10 to 20° C. The condensate exiting the heat exchanger 106 was split into a condensate product stream 107A and a condensate recycle stream 107B. The mass ratio of the condensate recycle stream 107B to the condensate product stream 107A may be from 10:1 to 20:1, depending on the temperature of the streams 107A and 107B, and in this particular example was 20:1. The condensate product stream 107A was collected.

An antifoaming agent 108 was added to the condensate recycle stream 107B. In this example, this was performed before the condensate recycle stream 107B was added into the condenser 102 (as discussed below). The antifoaming agent was Silfoam SE 47, as defined in Example 1.

The mixture of the condensate recycle stream 107B and the antifoaming agent 108 was added into the condenser 102.

The mixture 107B, 108 may be split into at least two streams 109A, 109B. The respective streams 109A, 109B may be added to the condenser 102 at different parts of the condenser 102. For example, the mixture 107B, 108 may be added at or towards the top of the condenser 102, e.g. above the packing material 103 (as indicated by stream 109A in FIG. 2). For example, the mixture 107B, 108 may be added at or towards the bottom of the condenser, e.g. below the packing material 103 (as indicated by stream 109B in FIG. 2).

The mixture 107B, 108 (109A) added through the top of the condenser 102 flowed downwards, through the packing material 103, under gravity. In this way, the mixture 107B, 108 (109A) flowed in countercurrent to the product gas 101 and promoted effective and efficient condensation of the product gas 101 in the condenser 102, while preventing excessive foaming. The packing material 103 in the condenser 102 increased the contact area between the mixture 107B, 108 (109A) and the product gas 101. The packing material was VFF Novalox-25-M AISI 316, although it will be appreciated that different packing materials may be used. The condenser 102 and the condensate recycling were operated continuously.

The amount of antifoaming agent 108 added to the condenser was approximately 0.2 mL per kg of glucose syrup feedstock solution added to the thermolytic fragmentation condenser. If no antifoaming agent 108 is used, an excessive amount of foam is formed, which can flood and/or block the condenser 102 and/or other equipment, resulting in reduced performance or equipment failure. The present invention addresses this problem.

It will be understood that the manner in which the antifoaming agent 108 is combined with the condensate may be varied. It is to be understood that the condensation process may be carried out in various different ways. For example, the manner in which condensate is recycled, and the nature and connectivity of the equipment used, may be varied. For example, various other equipment such as filters and other unit operations may be used. For example, alternative or additional cooling means may be used, e.g. addition of cooling liquid to the bottom of the condenser, underneath the packing material. It is also to be understood that control and monitoring may be exercised in various different ways. In this particular example, the liquid level at the bottom of the condenser 102 was the controlled variable. This can be achieved in various ways, e.g. by adjusting the amount of the mixture 107B, 108 recycled into the condenser 102 (e.g. by adjusting the flow velocity of the mixture 107B, 108 recycled into the condenser and/or the ratio of the streams 107A, 107B); and/or by adjusting the amount of the vapour phase oxygenate mixture entering the condenser 102.

Those skilled in the art will appreciate that the unit operations and any other features between formation of the fragmentation product 101 and the condensation process may be varied.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

The aspects and embodiments mentioned herein may be combined as is known to the person skilled in the art, and at least as given below:
Embodiment 1. A process for the at least partial condensation of an oxygenate mixture, the process comprising the steps of:
   (a) providing a vapour phase oxygenate mixture obtained from fragmentation of an aqueous solution of carbohydrates;
   (b) performing on the vapour phase oxygenate mixture an at least partial condensation to provide a condensate; and
   (c) combining an antifoaming agent and the condensate.
Embodiment 2. A process according to embodiment 1 wherein the vapour phase oxygenate mixture is derived from biomass.
Embodiment 3. A process according to embodiment 1 or 2 wherein the vapour phase oxygenate mixture comprises C1-C3 oxygenates.
Embodiment 4. A process according to any one of embodiments 1-3 wherein the carbohydrates of the aqueous solution of carbohydrates are selected from monosaccharides, disaccharides, or mixtures thereof.
Embodiment 5. A process according to any one of embodiments 1-4 wherein the carbohydrates of the aqueous solution of carbohydrates are at least glucose.
Embodiment 6. A process according to any one of embodiments 1-5 wherein the carbohydrates of the aqueous solution of carbohydrates comprise at least 40 wt. % of monosaccharides based on the total amount of carbohydrates.
Embodiment 7. A process according to any one of embodiments 1-6 wherein the antifoaming agent comprises one or more active ingredients independently selected from an alcohol, an ether, a carboxylic acid, an ester, a silicone, a silica, an oil, a wax, and an acrylate.
Embodiment 8. A process according to embodiment 7 wherein the one or more active ingredients is independently selected from an alcohol, such as a C1-C12 alcohol, or a C1-C6 alcohol, or a C1-C4 alcohol.
Embodiment 9. A process according to embodiment 7 or 8, wherein the antifoaming agent substantially comprises, consists essentially of, or consists of, the one or more active ingredients.
Embodiment 10. A process according to embodiment 7 wherein the one or more active ingredients is independently selected from a silicone, such as a polydimethylsiloxane.
Embodiment 11. A process according to any one of embodiments 7-10, wherein the antifoaming agent has a total active ingredient content of from 0.0000001 wt. % to 100 wt. %, based on the total weight of the antifoaming agent.
Embodiment 12. A process according to any one of embodiments 1-11 wherein the combining the antifoaming agent and the condensate comprises adding the antifoaming agent to the condensate.
Embodiment 13. A process according to embodiment 12 wherein the antifoaming agent is added to the condensate at a rate of from 0.0000001 mL/hour to 200 mL/hour.
Embodiment 14. A process according to any one of embodiments 1-13 wherein the the fragmentation of the aqueous solution of carbohydrates to provide the vapour phase oxygenate mixture comprises adding the aqueous solution of carbohydrates to a fragmentation reactor and subjecting the aqueous solution of carbohydrates to thermolytic fragmentation.
Embodiment 15. A process according to any one of embodiments 1-14 wherein the mass ratio of the antifoaming agent to the condensate in the combined antifoaming agent and condensate is from 1:1000000 to 1:1.
Embodiment 16. A process according to any one of embodiments 1-15 wherein in step (b) the vapour phase oxygenate mixture is at least partially condensed at a temperature of from 0 to 150° C.
Embodiment 17. A process according to any one of embodiments 1-16 wherein the performing on the vapour phase oxygenate mixture an at least partial condensation comprises:
   feeding an input stream comprising the vapour phase oxygenate mixture through a condenser to provide the condensate and an output stream.
Embodiment 18. A process according to embodiment 17 wherein the performing on the vapour phase oxygenate mixture an at least partial condensation comprises:
   recycling at least part of the output stream into the input stream.
Embodiment 19. A process according to embodiment 17 or 18 wherein the performing on the vapour phase oxygenate mixture an at least partial condensation comprises:
   splitting the condensate into at least a condensate product stream and a condensate recycle stream, and adding the condensate recycle stream to the condenser.
Embodiment 20. A process according to embodiment 19 wherein the combining the antifoaming agent and the condensate comprises adding the antifoaming agent to the condensate recycle stream to form a mixture and adding the mixture to the condenser.
Embodiment 21. A process according to embodiment 20 wherein the mixture added to the condenser has a temperature of from 0 to 30° C.
Embodiment 22. A process according to any one of embodiments 1-21 wherein the process is a continuous process.
Embodiment 23. A process according to any one of embodiments 1-22 comprising the further step of recovering the condensate.
Embodiment 24. A process according to any one of embodiments 1-23 wherein the fragmentation of the aqueous solution of carbohydrates is thermolytic fragmentation.
Embodiment 25. A process according to any one of embodiments 1-24 wherein step (b) is performed on an oxygenate mixture obtained directly from fragmentation of an aqueous solution of carbohydrates.
Embodiment 26. A system configured to at least partially condense an oxygenate mixture, the system comprising:
   (a) a fragmentation reactor configured to fragment an aqueous solution of carbohydrates to provide a vapour phase oxygenate mixture;
   (b) a condenser configured to at least partially condense the vapour phase oxygenate mixture to provide a condensate; and
   (c) a unit configured to combine an antifoaming agent and the condensate.
Embodiment 27. A system according to embodiment 26 wherein the fragmentation reactor is a selected from: a thermolytic fragmentation reactor; and a fluidised bed reactor such as a bubbling bed reactor, a turbulent bed reactor, or a riser-type reactor.
Embodiment 28. A system according to embodiment 26 or 27 wherein the fragmentation reactor comprises: a feed inlet; and a product outlet; a riser; and a fluidisation gas inlet.
Embodiment 29. A system according to any one of embodiments 26-28 comprising:
   a separator configured to separate particulate matter from the vapour phase oxygenate mixture.

1-23. (canceled)

24. A process for the at least partial condensation of an oxygenate mixture, the process comprising the steps of:
(a) providing a vapour phase oxygenate mixture obtained from fragmentation of an aqueous solution of carbohydrates;
(b) performing on the vapour phase oxygenate mixture an at least partial condensation to provide a condensate; and
(c) combining an antifoaming agent and the condensate.

25. The process according to claim 24, wherein the vapour phase oxygenate mixture is derived from biomass.

26. The process according to claim 24, wherein the vapour phase oxygenate mixture comprises C1-C3 oxygenates.

27. The process according to claim 24, wherein the carbohydrates of the aqueous solution of carbohydrates are selected from monosaccharides, disaccharides, or mixtures thereof.

28. The process according to claim 24, wherein the carbohydrates of the aqueous solution of carbohydrates comprise at least 40 wt. % of monosaccharides based on the total amount of carbohydrates.

29. The process according to claim 24, wherein the antifoaming agent comprises one or more active ingredients independently selected from an alcohol, an ether, a carboxylic acid, an ester, a silicone, a silica, an oil, a wax, and an acrylate.

30. The process according to claim 29, wherein the antifoaming agent has a total active ingredient content of from 0.0000001 wt. % to 100 wt. %, based on the total weight of the antifoaming agent.

31. The process according to claim 24, wherein the combining the antifoaming agent and the condensate comprises adding the antifoaming agent to the condensate.

32. The process according to claim 31, wherein the antifoaming agent is added to the condensate at a rate of from 0.0000001 mL/hour to 200 ml/hour.

33. The process according to claim 24, wherein the the fragmentation of the aqueous solution of carbohydrates to provide the vapour phase oxygenate mixture comprises adding the aqueous solution of carbohydrates to a fragmentation reactor and subjecting the aqueous solution of carbohydrates to thermolytic fragmentation.

34. The process according to claim 24, wherein the mass ratio of the antifoaming agent to the condensate in the combined antifoaming agent and condensate is from 1:1000000 to 1:1.

35. The process according to claim 24, wherein in step (b) the vapour phase oxygenate mixture is at least partially condensed at a temperature of from 0 to 150° C.

36. The process according to claim 24, wherein the performing on the vapour phase oxygenate mixture an at least partial condensation comprises:
feeding an input stream comprising the vapour phase oxygenate mixture through a condenser to provide the condensate and an output stream.

37. The process according to claim 36, wherein the performing on the vapour phase oxygenate mixture an at least partial condensation comprises:
recycling at least part of the output stream into the input stream.

38. The process according to claim 36, wherein the performing on the vapour phase oxygenate mixture an at least partial condensation comprises:
splitting the condensate into at least a condensate product stream and a condensate recycle stream, and adding the condensate recycle stream to the condenser.

39. The process according to claim 38, wherein the combining the antifoaming agent and the condensate comprises adding the antifoaming agent to the condensate recycle stream to form a mixture and adding the mixture to the condenser.

40. The process according to claim 24, wherein step (b) is performed on an oxygenate mixture obtained directly from fragmentation of an aqueous solution of carbohydrates.

41. A system configured to at least partially condense an oxygenate mixture, the system comprising:
(a) a fragmentation reactor configured to fragment an aqueous solution of carbohydrates to provide a vapour phase oxygenate mixture;
(b) a condenser configured to at least partially condense the vapour phase oxygenate mixture to provide a condensate; and
(c) a unit configured to combine an antifoaming agent and the condensate.

42. The system according to claim 41, wherein the fragmentation reactor is a selected from a thermolytic fragmentation reactor and a fluidised bed reactor.

43. The system according to claim 41, wherein the fragmentation reactor comprises:
a feed inlet;
a product outlet;
a riser; and
a fluidisation gas inlet.

44. The system according to claim 41, comprising:
a separator configured to separate particulate matter from the vapour phase oxygenate mixture.
